(19) United States
(12) Patent Application Publication
(10) Pub. No.: US 2010/0233285 A1
(43) Pub. Date: Sep. 16, 2010

(54) HONEY BASED COMPOSITIONS OF A CONSISTENCY THAT CAN BE DELIVERED TO THE RESPIRATORY SYSTEM

(75) Inventors: Mark Shane Stuart, Hamilton (NZ); Duncan George Mackintosh, Hamilton (NZ); Tania Leeanne Smith, Hamilton (NZ); Fiona Marie Miller, Hamilton (NZ)

Correspondence Address: SHERIDAN ROSS PC, 1560 BROADWAY, SUITE 1200, DENVER, CO, 80202, US

(73) Assignee: WAIKATOLINK LIMITED, Hamilton (NZ)

(21) Appl. No.: 12/519,002

(22) PCT Filed: Dec. 14, 2007
(86) PCT No.: PCT/NZ07/00365

§ 371 Date: Dec. 11, 2009

(30) Foreign Application Priority Data
Dec. 14, 2006 (NZ) 547946

(51) Int. Cl. A61K 35/64; A61P 31/16; A61P 31/04

(52) U.S. Cl. 424/537

(57) ABSTRACT

A method of treating a respiratory disease in an animal, characterised by the step of administering a composition of a consistency that can be delivered to the respiratory system of the animal, wherein the composition contains a bio-active fraction of honey.

### TECHNICAL FIELD

This invention relates to a treatment composition.

In particular this invention relates to a honey based treatment composition for the treatment of viral infections.

### BACKGROUND ART

Animals suffer from a large number of viral infections; these are particularly common in the respiratory system.

Common respiratory viruses in humans include influenza and the common cold.

There are two major forms of viruses, those with envelopes wherein the virus has an outer membrane or a viral envelope which helps the virus enter the host cell, or those with RNA genomes and no envelope.

Viruses are very difficult to treat for the following reasons.

Firstly, unlike most bacterial infections which lead to independent existence outside host cells, viruses are intracellular parasites. Isolated viruses are unable to replicate, being simply DNA or RNA within (or not) a protein envelope, viruses therefore require host cell mechanisms to replicate.

It is therefore extremely difficult to find compounds that can target and selectively block or prevent viral replication without interference to the normal cellular processes or significant toxicity to the host.

Due to the intercellular nature of the viruses many chemicals and compounds which are used to treat bacterial or other infections are too harsh, too toxic and can lead to permanent damage or death of the host cell, especially at the concentrations which may be required. Therefore they are undesirable for the prevention or treatment of viral infections.

Another reason is that each type of virus can only infect and parasitize a limited range of host cells, due to the recognition required between receptor cells on the host cell and the virus. For example the human common cold generally only infects the cells lining the upper respiratory tract. Any treatments, especially using targeted drugs, which may be harmful therefore also, need to be targeted to the infected cells. This can be difficult.

A further reason that some viral infections can be difficult to prevent or treat is that some viruses are highly adaptable and readily mutate. This can invalidate specific drugs or vaccines which may have been targeted to the original virus or viral sequence.

Many antiviral drugs are targeted specifically towards the virus, and preventing the replication of same. Antiviral drug design has two major branches, adapting existing effective drugs to determine if analogues of these are also effective, and rational drug design, a concept whereby a specific feature of the virus is targeted and a drug designed which can interact with this feature.

These drugs can be expensive, and as they are highly specific, are only useful once the virus causing the infection has been identified. They can also have numerous side effects. These can be undesirable, especially when the patient is a pregnant woman, a child or an elderly person.

Common antiviral vaccines are made from dead portions of the virus; however in some cases live vaccines are used. Live vaccines often use a very small amount of the virus and/or maybe an attenuated virus to prevent infection. Many people are worried or will not have vaccines due to the perceived risk of infection, this is especially the case with live vaccines.

There is a wide range of application techniques of administering antiviral drugs to patients.

Vaccines have the risk of causing a low-level (or full-blown) infection as a result of administering the virus (especially live vaccines) to try to get the body to build up specific antibodies for that virus.

Some common examples of application techniques include: injections, tablets, capsules, and absorption through the skin. All these methods have significant disadvantages.

Injections can be painful and invasive. Many people also have an aversion or phobia to needles which can hinder this technique of antiviral drug delivery.

Tablets or capsules are another common application method; however some people have aversions to swallowing tablets or capsules, especially young children. Often the very young, elderly or sick have difficulties swallowing, therefore finding capsules or tablets difficult to handle.

Tablets or capsules may also be unsuitable for delivery of compounds as they may render inactive by normal digestive processes.

The effectiveness of using tablets and capsules in administering antiviral drugs may also depend on the site of the infection.

Tablets and capsules are also not suitable for people or patients suffering nausea, for example cancer patients or people with travel sickness as they may vomit up the tablets and capsules and therefore not gain the benefits of same.

Suppositories for rectal or vaginal administration are not well liked or accepted by patients who have any other option available to them.

A method of treating viral infections, along with bacterial infections has previously been disclosed using plant essential oils.

US 2004/0009245 discloses a method of treating SARS, tuberculosis and other respiratory infections by inhalation of the vapours from one hundred percent botanical essential oils. Essential oils such as eucalyptus oil (Eucalyptus globules) and tea tree oil (Melaleuca alternifolia) are disclosed as containing significant antibacterial, antifungal properties.

However, there are significant disadvantages in requiring a vapour form for inhalation. These include the fact that the oils must be volatile, having a volatile substance increases the difficulty with which the dosage can be controlled, and volatile vapours may also be likely to cause irritation.

It would therefore be beneficial if there were available a compound which can treat respiratory disease in particular especially microbial diseases including viral diseases in the respiratory system, which is substantially a natural product and easy to administer.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method of treating a respiratory disease in an animal,

characterised by the step of
administering a composition of a consistency that can be delivered to the respiratory system of the animal,
wherein the composition contains a bio-active fraction of honey.

According to another aspect of the present invention there is provided a composition for treating a respiratory disease in an animal including a therapeutically effective amount of a bioactive honey fraction

characterised in that the composition is of a consistency that can be delivered to the respiratory system of the animal.

According to another aspect of the present invention there is provided a drug delivery device, including

a composition of a consistency that can be delivered to the respiratory system of an animal and which contains a bioactive honey fraction.

In preferred embodiments the present invention is directed to microbial respiratory diseases and in particular viral respiratory diseases. However this should not be seen as limiting.

In some embodiments the composition may also include other components, including, but not limited to various types of honey, fractions or components from manuka, or other types of honey, or any pharmaceutically acceptable components.

Preferably the honey fraction comes from manuka honey, provided there is in the fraction sufficient bio-activity that can be useful in the treatment of respiratory diseases.

Manuka honey is preferred as to date this has been shown to be the honey that has the greatest amount of bio-activity. This bio-activity has been associated in manuka honey with the term UMF.

Throughout this specification the term UMF should be taken as meaning Unique Manuka Factor.

Honey has long been used as an antibacterial agent. The major cause of the antibacterial activity is due to hydrogen peroxide that is produced in honey by the enzyme glucose oxidase. Manuka honey has been found to possess an amount of activity that is in addition to this antibacterial activity. This additional activity is known as the non-peroxide activity and is commercially know as Unique Manuka Factor, UMF.

Even though UMF is measured with reference to antibacterial activity, the inventors have found that this same factor is effective against viruses as well.

As can be appreciated, whole honey is a very viscious substance and would be very difficult to introduce into a respiratory system of an animal without significant physiological discomfort or even damage.

Thus, if whole honey was to be used in a treatment composition, it would need to be solubilised to reduce its viscosity and able to be delivered to the respiratory system. The action of solubilising dilutes the active portion of the honey, thus rendering the composition less effective. To be as equally effective as the non solubilised whole honey, a considerable greater volume is required.

A greater volume in its own right is undesirable with regard to delivery to the respiratory system. Ideally the respiratory system should receive as little liquid as possible, just enough to introduce the active component but not so much as to flood the system of the animal being treated.

Further, the applicants have found that the active fraction of honey is contained to a very small volume of the whole honey.

It can therefore be seen to be desirable to incorporate into the composition a fraction which is high in activity and which does not have the volume or viscosity of whole honey.

Fortunately, the applicants have determined that it is possible to obtain a small fraction of honey which makes up less than 1% of the dry weight yet contains virtually all the non-peroxide bio-activity and can be isolated in the bulk of manuka honey as a UMF containing fraction.

A description of how that fraction can be obtained is provided in New Zealand Patent No. 533368 and equivalent applications derived from PCT/NZ2005/000118.

This fraction only has a modicum of monosaccharides if at all and therefore is non-viscous and highly potent for its volume. Whole honey is typically comprised of more than 80% by volume of monosaccharides.

Using fractionated manuka honey fractions, provides greater efficiency in treating viral respiratory diseases and allow lower volumes of honey to be utilised aiding delivery of concentrated active.

In a preferred embodiment the manuka honey composition may also include a carrier or other pharmaceutically acceptable compound.

In a preferred embodiment the other pharmaceutically acceptable compound(s) may include compounds which enable the nebulisation or atomisation or the manuka honey composition.

Throughout this specification the term nebulisation or atomization should be taken as processing or reducing the manuka honey composition into minute particles or a fine spray, which is sufficiently fine to be absorbed into the respiratory system of the patient. For example, the honey fraction is in a mist form that can be carried in the air to the lungs.

In one preferred embodiment the pharmaceutically acceptable compound may also be a natural compound.

In a preferred embodiment the addition of a carrier or thinning agent may provide the composition with the desired consistency.

In a preferred embodiment the carrier or thinning agent may be water or saline. However, these should not be seen as limiting, one skilled in the art would be aware that a wide range of agents or compounds may be utilised to provide the desired consistency.

It should be appreciated that with the present invention being a fraction of total honey, it is much easier solubilise with thinning agents such as water or saline than if a sugar laden composition had been used.

Throughout this specification the term thinning agent should be taken to mean a compound or composition which when added to the manuka honey composition provides a substantially more fluid consistency.

In a preferred embodiment the fortified honey composition may be in a substantially liquid form. However this should not be seen as limiting as a fine powder or other suitable form may also be utilised.

Having the manuka honey composition in a liquid formulation allows it to easily be delivered via a nose spray or other spray device to the respiratory area to be treated.

In an alternative embodiment the honey composition may be in the form of a fine powder which then is quickly and easily delivered to the respiratory system.

Again, the honey in the form of a powder can be much readily achieved if the majority of the monosaccharides have been removed from the honey to give a fraction as described previously.

In a preferred embodiment the manuka honey composition may also include pharmaceutically acceptable compound which allows the manuka honey composition to be administered through the nasal passage, but not available for adsorption until it reaches the lungs. This is specifically the case for viral respiratory diseases of the lungs.

In other embodiments the manuka honey composition may also include any other pharmaceutically acceptable compounds. For example methanol.

In a preferred embodiment the virus that manuka honey composition is used to treat may be the influenza virus. However, this should not be seen as limiting as the manuka honey composition of the present invention may be utilised to treat any viral respiratory disease.

A key aspect to the present invention is that the inventor has found that a honey fraction containing UMF as previously discussed is effectively anti-viral. And in particular, effective in relation to viral respiratory diseases such as influenza.

The manuka honey composition of the present invention is a natural product. This is highly advantageous in the current market where patients are looking for healthy and non-chemical alternatives.

The manuka honey composition of the present invention may be applied after the infection has colonised the respiratory system. This could be a substitute for the need for vaccines. Vaccines, for such viral infections as the flu are specific to one strain, which is believed will be most prevalent in the near future. However, often this strain is not, leading to unnecessary vaccines. This is especially the case for young children or those who have an aversion to needles and injections.

Alternatively the invention may be used as a preventative measure prior to any symptoms of the disease manifesting.

As the manuka honey composition combats the infection, there is also no need (perceived or otherwise) for the patient having the virus artificially introduced as a step for the body to build up antibodies.

The manuka honey composition therefore has all the advantages of previous natural antiviral products, such as those derived from essential oils (*US 2004/0009245). However the essential oil products are in a volatile form. These can have significant disadvantages. For example volatile sprays (such as those administered to the nasal cavity, can lead to irritation and irreversible damage to the nasal cavity from chronic application of nasal dosage.

It is also difficult to control the dosage volume when in a volatile form, often leading to over or under administration. This can decrease the effectiveness of the composition, and lead to increased infection times.

In a preferred embodiment the manuka honey composition may be non-volatile.

In a preferred embodiment the term non-volatile should be taken as meaning that the composition does not evaporate quickly, and is stable. This is an advantage in that it allows the composition to be easily stored and used.

As well as the above, the present invention provides a number of significant advantages over the previous concept of using volatile oils to provide an anti-viral effect. These include the following:
it is a natural product, thereby reducing the chemicals utilised and increasing consumer acceptance,
the composition uses only a small fraction of honey, but one with high potency or bio-activity so that a low volume effective natural dose can be given.
being non-volatile the honey composition does not have to be prepared into a gaseous or volatile form, this decreases the manufacturing and production costs,
it is simpler to administer, and suitable for people of all ages or abilities,
it can be directly applied, therefore having a quicker and more effective response,
it can be provided in a more concentrated form,
the dosage rate can be easily controlled.

### BRIEF DESCRIPTION OF DRAWINGS

Further aspects of the present invention will become apparent from the ensuing description which is given by way of example only and with reference to the accompanying drawings in which:

FIG. 1: illustrates HPLC refractive index analysis of manuka honey using ligand exchange and size exclusion chromatography.

FIG. 2: illustrates enlarged base line region of a HPLC refractive index analysis of manuka honey by ligand exchange and size exclusion chromatography.

### BEST MODES FOR CARRYING OUT THE INVENTION

The following examples provide initial results as to the type of fraction that can be used to obtain effective anti-viral activity.

### Example 1

This is a typical fraction that can be used with a composition in accordance with the present invention.

High performance liquid chromatography (HPLC) was conducted on a Waters HPLC system using a 515 HPLC pump, a 2410 refractive index detector, a 996 photodiode array detector and Millennium operating software.

In initial studies Shodex™ Sugar KS800 series columns were found to provide the best separation out of all the columns used. Here, Shodex™ Sugar KS801 and KS 802 were used in series to fractionate the honey samples by combined size exclusion and ligand exchange chromatography.

The KS801 was in the sodium form with an exclusion limit of 10³. KS802 was also in the sodium form and had an exclusion limit of 10⁴. Both were packed with styrene divinylbenzene. The operating temperature used was initially 80° (with a flow rate of 1 mL/min) as suggested by the manufacturer. The eluant was Milli-Q water.

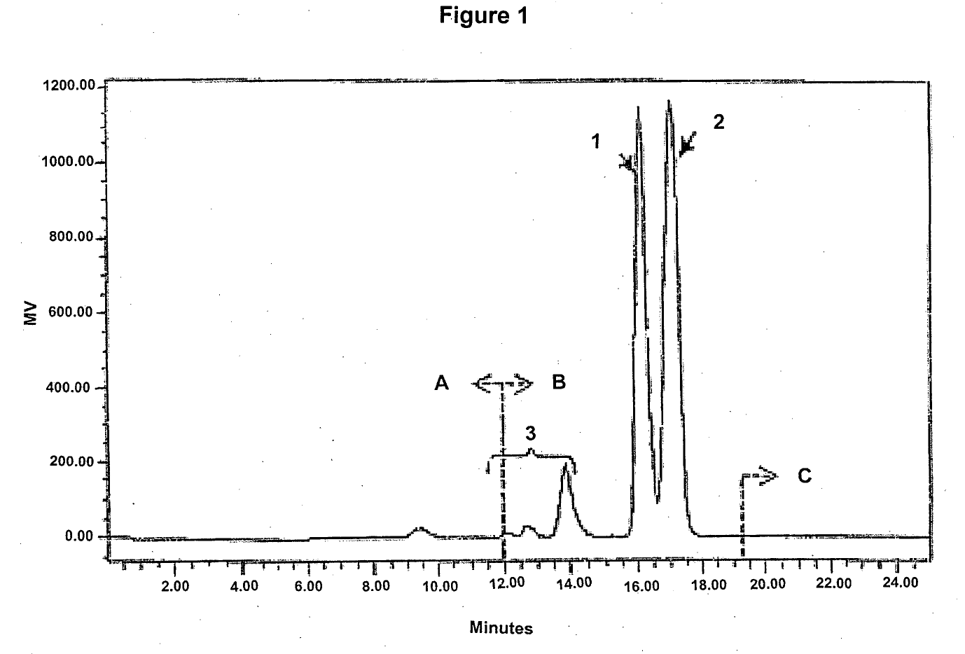

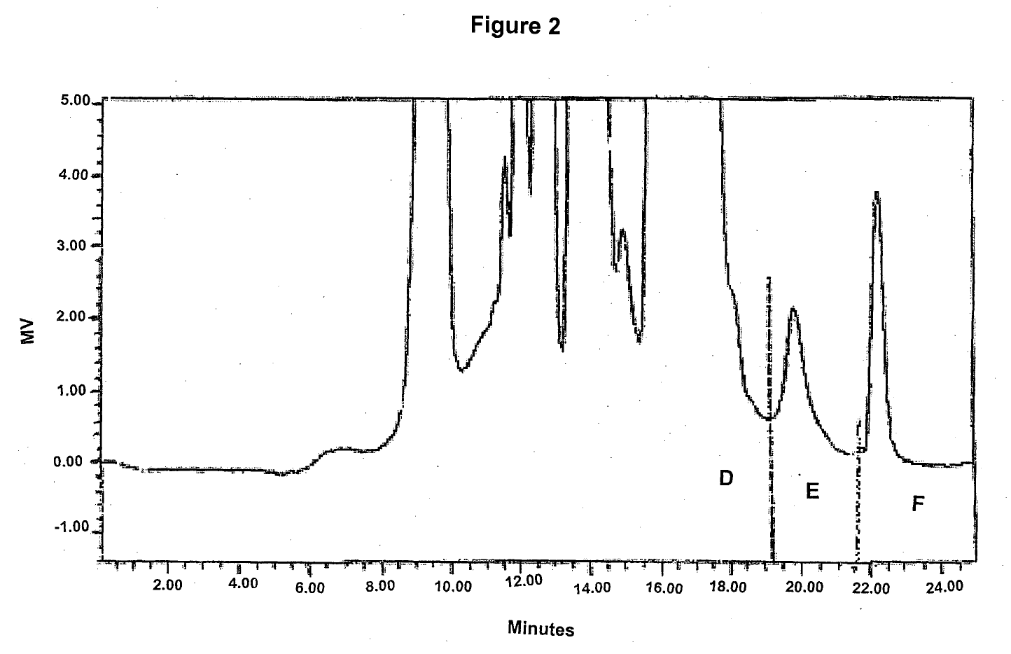

Honey samples of 20 mg/20 μL injection were loaded onto the KS800 series HPLC columns. FIGS. 1 and 2 show the plots obtained with refractive index detection. Fractions were collected for antibacterial assay from 20 injections.

In FIG. 1, the fraction A was collected from 0 to 12 minutes, fraction B was collected between 12 and 19.4 minutes, and fraction C was collected from 19.4 to 25 minutes. The plot shows the glucose (1) peak followed by the fructose (2) peak. An oligosaccharide (3) peak is also shown.

The antibacterial activity of the separate fractions was tested using the well diffusion technique using Staphylococcus aureus as the test culture.

Fractions collected from the HPLC for testing were evaporated under reduced pressure on a Büchi RE111 Rotovapor coupled with a Büchi 461 water bath at 40° C. The samples were then re-dissolved in a solution containing 200 μL of distilled water and 200 μL of catalase solution to ensure only non-peroxide activity was present.

Honey samples were tested in a concentration of 25% for antibacterial activity. The antibacterial assays were conducted using three replicates of the phenol standards ranging from 2% to 6% and three to five replicates of the samples being tested were introduced into recorded random wells in the agar plates.

The plates were incubated at 37° C. overnight allowing the bacteria to grow where possible. After incubation, digital calipers were used to measure the diameter of the area of inhibition around the wells.

The non-peroxide antibacterial activity of the honey was completely contained within fraction C (FIG. 1).

When focusing in on the baseline region of the HPLC plot, two peaks were visible in the active region of fraction C (FIG. 2). To determine which of these peaks was responsible for the activity, another scheme of fraction collection times was devised: fraction D 0 to 19.14 minutes, fraction E 19.4 to 21.7 minutes, and fraction F 21.7 to 25 minutes.

In these experiments, all the antibacterial activity was isolated in fraction E.

It is believed that this fraction would work well with the present invention.

This test was repeated a further two times and the same results obtained.

### Example 2

However, testing of the effectiveness of a number of other manuka honey fractions was undertaken in an Influenza Assay with respect to the strain Influenza A Puerto Rico A/PR/8/34, and to determine toxicity.

### Collection of Fractions:

The reverse phase fractionation column used was three Delta-Pak 018 cartridges (25 mm×100 mm, 15 μm particle size, 100A pore size) in series. This was fitted with a Delta C18 guard insert (25 mm×10 mm, 15 μm particle size, 100A pore size). Chromatography was performed at room temperature at a flow rate of 10 mL/min with high loadings of 0.5 g/mL into a 2 mL loop.

The inventors used only Milli-Q water. Due to the high flow rate used, detection was only possible using a wide-bore plumbed Waters 410 differential refractometer.

Fractions collected in water were freeze-dried in large evaporating dishes. Fractions collected in MeCN were concentrated under reduced vacuum and then drying was completed on the freeze drier.

The elutant from the column was collected in three main fractions, being:

| | | |
|---|---|---|
| Fraction A | 0.0 to 8.3 minutes | Early eluting material |
| Fraction B | 8.3 to 11.8 minutes | Sugars |
| Fraction C | 11.8 to 25.0 minutes | Later eluting material including UMF |
| | | but minimal sugar |

MeCN is a stronger solvent than water in reversed phase chromatography and so it can be used to flush any residual material from the column.

The column was initially run with Milli-Q water and fractions A and B were collected as previously outlined. At the cross-over from fraction B to fraction C (11.8 minutes) the pump was stopped, and the solvent swapped directly over to 100% MeCN.

The following samples were used during testing.

| Code | Sample |
|---|---|
| 12.5 | 12.5 UMF Manuka Honey |
| 12.5A | 12.5 UMF Manuka Honey Fraction A |
| 12.5B | 12.5 UMF Manuka Honey Fraction B |
| 12.5C | 12.5 UMF Manuka Honey Fraction C |
| 15 | 15.0 UMF Manuka Honey |
| 15.0A | 15.0 UMF Manuka Honey Fraction A |
| 15.0B | 15.0 UMF Manuka Honey Fraction B |
| 15.0C | 15.0 UMF Manuka Honey Fraction C |
| Cl | Clover Honey (0 UMF) |

The fraction from 4.4382 g/˜3.1233 mL of honey was collected by HPLC in a large volume of milliQ water (about 400 mL) and roto-evaporated down to between 0.5 and 1 mL. The fractions were made up to 1.2 mL in a 10 mL measuring cylinder with milliQ water and then up to 3.1 mL with 0.9% NaCl solution (except for B fractions).

12.5B and 15.0B could not be roto-evaporated to less than 1 mL because of their high sugar content. They formed syrup and could not be reduced further.

The dilution with milliQ step was therefore skipped and the fractions were made up to 3.1 mL with 0.9% NaCl. The volumes of 12.5B and 15.0B before making up to volume with saline were 1.7 mL and 2.1 mL respectively.

The B fractions were the most concentrated and could be up to at least 80% sugar (mostly glucose and fructose) by volume. The other fractions are much less concentrated; estimate less than 10% sugar by volume.

After collection by HPLC the fractions were stored in a walk in fridge at 8-10° C.

After the fractions had been roto-evaporated and made up to the final volume they were stored in the freezer. Only one of the fractions froze so the fractions were moved to the walk in freezer at −10 to −15′C. Most fractions have frozen but a couple did not freeze.

The following table lists the volume of original sample and the volume of 0.9% NaCl added to make up to 3.1 ml volume in, to give an indication of concentration per mL (of samples used in testing). Some of the samples had a very high/high sugar content, these are noted.

The undiluted samples were also very viscous, which in some cases meant that dilution was required prior to filter sterilisation.

| Sample | | |
|---|---|---|
| ID | Concentration of sample | Notes |
| 12.5 | Undiluted | Very high sugar |
| | | content |
| 12.5A | 1.2 ml sample + 1.9 ml 0.9% NaCl = 3.1 ml | |
| 12.5B | 1.7 ml sample + 1.4 ml 0.9% NaCl = 3.1 ml | High sugar |
| | | content |
| 12.5C | 1.2 ml sample + 1.9 ml 0.9% NaCl = 3.1 ml | |
| 15.0 | Undiluted | Very high sugar |
| | | content |
| 15.0A | 1.2 ml sample + 1.9 ml 0.9% NaCl = 3.1 ml | |
| 15.0B | 2.1 ml sample + 1.0 ml 0.9% NaCl = 3.1 ml | High sugar |
| | | content |
| 15.0C | 1.2 ml sample + 1.9 ml 0.9% NaCl = 3.1 ml | |
| 21.1 | Undiluted | Very high sugar |
| | | content |
| 28.8 | Undiluted | Very high sugar |
| | | content |
| Cl | Undiluted | Very high sugar |
| | | content |

All samples were tested in an influenza bioassay as per the method of Faulkner et al (Vaccine, 2003).

Samples were filter sterilized prior to use to eliminate the risk of contamination in the assay (B fractions were not diluted prior to filter Sterilization).

1 ml aliquots of all the samples were filter sterilized and then used either neat or further diluted in sterile PBS before adding to the cell line.

Viscous, whole honey samples were first re-suspended in PBS to a final concentration of 100 mg/ml then filter sterilized and used in the assay.

Results

Samples 12.5B, 12.5C, 15.0B and 15.0C show protective activity against viral infection at dilutions of 1/40, 1/40 and 1/8 respectively (Figure A).

Samples 12.5B and 15.0B were toxic to cells in the absence of virus at the highest concentration used in the assay (Figure B).

Due to the B samples not being diluted prior to sterilization their high sugar content may be cytotoxic at 1/8 dilution.

Raw samples were diluted 100 mg/ml (>>than 1/10 dilution) and then diluted 1/8 (to give >>1/80) so would not have seen the same cytotoxic effect because of sugar level.

Viscous samples were reconstituted at 100 mg/ml of product prior to filter sterilising and using in assay. Viscous unfiltered samples were also reconstituted at 100 mg/ml.

12.5B and 15.0B are both liquid samples and were filtered and then diluted 1/8, 1/40/1/200 and 1/1000. At 1/8 dilution both of these products were toxic to cells in the absence of virus. Both were non-toxic to cells at the 1/40 dilution and were also protective against viral infection at this dilution.

Samples number 12.5B, 12.50, 15.0B and 15.0C were able to reduce the ability of the flu virus to kill the cells.

It should be noted that Fraction C has considerably less volume and viscosity than Fraction B.

All other samples showed little or no activity (FIG. 3). All samples were compared to our in-house positive control, which is known to afford protection in this assay. We were unsure if the viscous samples were affected by filtration and if this was the reason for the negative result seen in the assay so we also tested them without filtration. No contamination occurred in the assay with unfiltered samples. When unfiltered, samples 12.5 and 28.8 were protective at a concentration of 12 mg/ml. None of the other unfiltered viscous samples were protective.

TABLE 1
Percentage of uninfected cells after addition
of diluted filtered viscous samples

| | |
|---|---|
| Sample | Percentage of Uninfected Cells |

| ID | 10 mg/ml | 5 mg/ml | 2.5 mg/ml | 1.25 mg/ml |
|---|---|---|---|---|
| 12.5 | 17 | 16 | 16 | 16 |
| 15.0 | 18 | 16 | 14 | 16 |
| 21.1 | 17 | 15 | 16 | 16 |
| 28.8 | 18 | 17 | 17 | 19 |
| Cl | 16 | 16 | 16 | 15 |

*Virus only 14% of cells uninfected

TABLE 2
Percentage of uninfected cells after addition
of diluted filtered liquid samples

| Sample | Percentage of Uninfected Cells | | | |
|---|---|---|---|---|
| ID | Dilution 1/8 | Dilution 1/40 | Dilution 1/200 | Dilution 1/1000 |
| 12.5A | 28 | 25 | 22 | 21 |
| 12.5B | 16 | 100 | 30 | 22 |
| 12.5C | 100 | 31 | 23 | 26 |
| 15.0A | 21 | 17 | 17 | 17 |
| 15.0B | 9 | 100 | 28 | 23 |
| 15.0C | 100 | 61 | 21 | 21 |

*Virus only 19% of cells uninfected

TABLE 3
Percentage of uninfected cells after addition
of diluted unfiltered viscous samples

| | |
|---|---|
| Sample | Percentage of Uninfected Cells |

| ID | 10 mg/ml | 5 mg/ml | 2.5 mg/ml | 1.25 mg/ml |
|---|---|---|---|---|
| 12.5 | 72 | 34 | 31 | 32 |
| 15.0 | 39 | 34 | 31 | 35 |
| 21.1 | 46 | 43 | 28 | 35 |
| 28.8 | 96 | 38 | 30 | 33 |
| Cl | 32 | 18 | 17 | 20 |

*Virus only 27% of cells uninfected

Aspects of the present invention have been described by way of example only and it should be appreciated that modifications and additions may be made thereto without departing from the scope of the appended claims.

1. A method of treating a respiratory disease in an animal, characterised by the step of
administering a composition of a consistency that can be delivered to the respiratory system of the animal,
wherein the composition contains an isolated fraction of honey having bio-activity.

2. A method as claimed in claim 1 wherein the honey is manuka honey.

3. A method as claimed in claim 1 wherein the bio-activity is that of UMF.

4. A method as claimed in claim 1, wherein the composition includes a carrier for the honey fraction.

5. A method as claimed in claim 4 wherein the carrier is water.

6. A method as claimed in claim 4 wherein the carrier is saline.

7. A method as claimed in claim 1, wherein the composition is administered via a nose spray.

8. A method as claimed in claim 1, wherein the composition is in the form of a powder.

9. A method as claimed in claim 1, wherein the respiratory disease is influenza.

10. A method as claimed in claim 1, wherein the composition is non-volatile.

11. A method as claimed in claim 1, wherein the fraction is obtained from the steps of
a) applying sample of manuka honey containing the fraction to a chromatography matrix in the format of a column;
b) retaining the sample on the matrix;
c) eluting the sample from the matrix with water; and
d) collecting the fraction, wherein the fraction is a UMF containing fraction;
and wherein the UMF containing fraction is substantially free of monosaccharide sugars.

12. A method as claimed in claim 11 wherein the matrix has a 15 μm particle size and a 100 Å. pore size.

13. A method as claimed in claim 11 wherein the UMF containing fraction has anti-bacterial activity and wherein the anti-bacterial activity of the UMF containing fraction is labile at a pH greater than 9.

14. A method as claimed in claim 11, wherein the anti-bacterial activity has the chromatagraphic characteristics described in Example 1.

15. A method as claimed claim 11, wherein the fraction has a retention time of 19.4 to 25 minutes when a sample (20 μL) of honey containing the UMF containing fraction is applied to Shodex™ Sugar KS-801 and KS-802 analytical columns in series and in the sodium form, operated at a temperature of 50° C. and eluted with Milli-Q water at a rate of 1 mL/min.

16. A method as claimed in claim 15 wherein the fraction has a retention time of 19.4 to 21.7 minutes.

17. A method as claimed claim 11, wherein the fraction has a retention time of 18.4 to 30 minutes when a sample (20 μL) of honey containing the UMF containing fraction is applied to Shodex™ KS2002 analytical column, operated at room temperature and eluted with Milli-Q water at a rate of 3 mL/min.

18. A method as claimed in claim 11, wherein the fraction has a retention time of 11.8 to 25 minutes when a sample (20 μL) of honey containing the UMF containing fraction is applied to Delta-Pak C18 analytical column, operated at room temperature and eluted with Milli-Q water followed by acetonitrile at a rate of 10 mL/min.

19. A composition for treating a respiratory disease in an animal comprising a therapeutically effect amount of an isolated honey fraction having bio-activity, wherein
the composition is of a consistency that can be delivered to the respiratory system of an animal.

20. A composition as claimed in claim 19, wherein the honey is manuka honey.

21. A composition as claimed in claim 19 wherein the bio-activity is that of UMF.

22. A composition as claimed in claim 19, wherein the composition includes a carrier for the honey fraction.

23. A composition as claimed in claim 22 wherein the carrier is water.

24. A composition as claimed in claim 22 wherein the carrier is saline.

25. A composition as claimed in claim 19, wherein the composition is administered via a nose spray.

26. A composition as claimed in claim 19, wherein the composition is in the form of a powder.

27. A composition as claimed in claim 19, wherein the respiratory disease is influenza.

28. A composition as claimed in claim 19, wherein the composition is non-volatile.

29. A composition as claimed in claim 19, wherein the fraction is obtained from the steps of
a) applying sample of manuka honey containing the fraction to a chromatography matrix in the format of a column;
b) retaining the sample on the matrix;
c) eluting the sample from the matrix with water; and
d) collecting the fraction, wherein the fraction is a UMF containing fraction; and wherein
the UMF containing fraction is substantially free of monosaccharide sugars.

30. A composition as claimed in claim 29 wherein the matrix has a 15 μm particle size and a 100 Å. pore size.

31. A composition as claimed in claim 29 wherein the UMF containing fraction has anti-bacterial activity and wherein the anti-bacterial activity of the UMF containing fraction is labile at a pH greater than 9.

32. A composition as claimed in claim 29, wherein the anti-bacterial activity has the chromatographic characteristics described in Example 1.

33. A composition as claimed in claim 29, wherein the fraction has a retention time of 19.4 to 25 minutes when a sample (20 μL) of honey containing the UMF containing fraction is applied to Shodex™ Sugar KS-801 and KS-802 analytical columns in series and in the sodium form, operated at a temperature of 50° C. and eluted with Milli-Q water at a rate of 1 mL/min.

34. A composition as claimed in claim 33 wherein the UMF containing fraction has anti-bacterial activity and a retention time of 19.4 to 21.7 minutes.

35. A composition as claimed in claim 29, wherein the fraction has a retention time of 18.4 to 30 minutes when a sample (20 μL) of honey containing the UMF containing fraction is applied to Shodex™ KS2002 analytical column, operated at room temperature and eluted with Milli-Q water at a rate of 3 mL/min.

36. A composition as claimed in claim 29, wherein where the fraction has a retention time of 11.8 to 25 minutes when a sample (20 μL) of honey containing the UMF containing fraction is applied to Delta Park C18 analytical column, operated at room temperature, and eluted with Milli-Q water followed by acetonitrile at a rate of 10 mL/min.

37. A drug delivery device including
a composition having a consistency that can be delivered to the respiratory system in an animal and which contains an isolated honey fraction having bio-activity.

38-40. (canceled)
